(19) United States
(12) Patent Application Publication
(10) Pub. No.: US 2008/0050391 A1
(43) Pub. Date: Feb. 28, 2008

(54) Method for Treating Autoimmune Diseases With Antibodies

(76) Inventors: Alan H. Lazarus, Toronto (CA); Seng Song, Toronto (CA); Andrew R. Crow, Scarborough (CA); Vinayakumar Siragam, Toronto (CA); Davor Brinc, Toronto (CA); John Freedman, Toronto (CA)

Correspondence Address: JENKINS, WILSON, TAYLOR & HUNT, P. A., 3100 TOWER BLVD., Suite 1200, DURHAM, NC, 27707, US

(21) Appl. No.: 10/594,826

(22) PCT Filed: Mar. 30, 2005
(86) PCT No.: PCT/CA05/00472

§ 371 Date: Jul. 17, 2007

Related U.S. Application Data
(60) Provisional application No. 60/558,080, filed on Mar. 30, 2004.
(60) Provisional application No. 60/613,712, filed on Sep. 29, 2004.

(51) Int. Cl. A61K 39/395; A61K 39/00; A61P 19/02; A61P 7/00

(52) U.S. Cl. 424/157.1; 424/130.1

(57) ABSTRACT

A method for treating autoimmune diseases in a mammal which method comprises administering to the mammal an effective amount of at least one antibody specific for a soluble antigen is provided. Furthermore, a novel mechanism of action has been established in accordance with the present invention for antibody-based treatment regimes for autoimmune disease, including but not limited to anti-CD44 and soluble antigen specific antibody treatment regimes.

### TECHNICAL FIELD

This application relates to the treatment of autoimmune diseases using antibodies. More preferably, the present invention relates to treatment of autoimmune diseases with soluble antigen-specific antibodies.

### BACKGROUND OF THE INVENTION

Immune thrombocytopenic purpura (ITP) is an autoimmune disease characterised by platelet clearance mediated by pathogenic anti-platelet antibodies. It has been previously suggested that this platelet clearance is mediated by Fcγ receptor (FcγR)-bearing macrophages in the reticuloendothelial system (RES). While intravenous immunoglobulin (IVIg) is widely used in the treatment of ITP as well as in a wide variety of chronic autoimmune and inflammatory diseases, its mechanism of action is not yet fully elucidated. Possible mechanisms of action include inhibition of RES function, anti-idiotype antibodies and immunomodulation. In murine models of ITP, it has been demonstrated that IVIg ameliorates ITP by a mechanism completely dependent upon the expression of the inhibitory FcγRIIB. In humans, there is also evidence that IVIg increases the level of expression of FcγRIIB. In addition, it has been previously reported that the clinical effects of IVIg as well as monoclonal mimetics of IVIg both ameliorate murine ITP in a manner that correlates with RES blockade. This ‘competitive’ RES blockade has long been considered to be the primary mechanism whereby IVIg ameliorates ITP.

The present study was undertaken to investigate if antibodies to soluble antigens could inhibit autoimmune diseases.

### SUMMARY OF THE INVENTION

According to the present invention, a novel method for treating an autoimmune disease is provided. Furthermore, a novel mechanism of action has been established in accordance with the present invention for antibody-based treatment regimes for autoimmune disease, including, but not limited to anti-CD44 and soluble antigen specific antibody treatment regimes.

In one embodiment of the invention there is provided a method for treating autoimmune diseases in a mammal which method comprises administering to the mammal an effective amount of at least one antibody specific for a soluble antigen.

Different types of autoimmune diseases be treated by the method of the present invention. According to the present invention, an autoimmune disease includes, but is not limited to Immune thrombocytopenia, Immune cytopenia, Idiopathic thrombocytopenic purpura (ITP), Neuropathy, Chronic inflammatory demyelinating polyneuropathy (CIDP), Guillain-Barre syndrome (GBS), Kawasaki's disease, Dermatomyositis, SLE, Myasthenia gravis, Post-transfusion purpura, Rheumatoid arthritis, Inflammatory arthritis, Eaton-Lambert syndrome, toxic epidermal necrolysis, and polymyositis.

In one embodiment, the treatment can be effected for a time and under conditions sufficient to inhibit platelet clearance, thereby treating or ameliorating an autoimmune disease such as immune thrombocytopenic purpura (ITP), for example. In a further embodiment, inflammatory arthritis can be prevented or ameliorated by the administration of antibodies to a soluble antigen in accordance with the present invention.

The soluble antigen can either be an endogenous or a foreign antigen. By foreign antigen it is meant an antigen that is not normally produced by the same individual or species. The antigen can be a non-functional/inert antigen. In an other embodiment the binding of the antibody to the antigen does not compromise the function of the antigen.

In an aspect of the invention the soluble foreign antigen and the antibody can be incubated together to form antibody-antigen complexes prior to administering the complexes to the mammal.

In another aspect of the invention, the endogenous soluble antigen can be obtained from the mammal and incubated with the antibody to form antibody-antigen complexes, the complexes being subsequently administered to the mammal. Alterntively, a soluble antigen may be injected into a mammal having a pre-existing antibody of interest specific to the soluble antigen, e.g. a mammal who has been previously immunised to tetanus toxin (any # of years earlier) may be administered an injection of soluble tetanus toxin according to an alternate embodiment of the present invention.

The antibody can be administered intravenously, interperitoneally, intradermally, intramuscularly, subcutaneously, orally or rectally.

In another embodiment of the invention, the soluble antigen can be associated with blood cells and the resulting antigen-cell complexes can be targeted by antibodies for inhibiting platelet clearance and thereby treating thrombocytopenia.

In another embodiment, an autoimmune disease treatment regime is provided to mediate a cellular response in dendritic cells, such as leukocytes, such that platelet clearance is slowed and/or inhibited, thereby treating or ameliorating an autoimmune disease.

In another aspect of the invention there is provided pharmaceutical compositions for treating autoimmune diseases such as arthritis and thrombocytopenia, comprising an effective amount of at least one antibody specific for a soluble antigen and/or for a soluble antigen associated with a blood cell.

In yet another aspect of the invention, an antibody to a soluble antigen may be used in the manufacture of a medicament for the treatment of an autoimmune disease.

In yet another aspect of the invention, we demonstrate herein that antibodies to soluble antigens can ameliorate ITP in an FcγRIIB-dependent manner. Antibody directed to the cell-associated antigen inhibited ITP in an FcγRIIB-independent manner. Taken together, these data demonstrate that IgG antibodies reactive with either a soluble or insoluble antigen can mimic the effects of IVIg. In addition, the mechanisms of action of these moieties are quite different: antibody reacted with soluble antigen may utilize the same pathway used by IVIg, i.e. an FcγRIIB-dependent pathway, whereas antibody reacted with a cell-associated antigen may work by additional and/or other mechanisms of action, and possibly by competitive RES inhibition.

### BRIEF DESCRIPTION OF THE DRAWINGS

Further features and advantages of the present invention will become apparent from the following detailed description, taken in combination with the appended drawings, in which:

FIGS. 1A and 1B illustrate the association of OVA on the surface of RBCs.

FIGS. 2A and 2B illustrates inhibition of thrombocytopenia by treating OVA-coupled RBCs with OVA-specific IgG.

FIGS. 3A, 3B and 3C illustrates amelioration of thrombocytopenia with antibodies reactive with soluble OVA (in combination with soluble OVA) ameliorate immune thrombocytopenia.

FIGS. 4A and 4B illustrates inhibition of RES function by antibodies reactive with soluble OVA (FIG. 4A) or OVA-RBCs (FIG. 4B).

FIG. 5 illustrates that antibodies reactive with soluble OVA or OVA-RBCs both ameliorate immune thrombocytopenia independent of complement activity.

FIGS. 6A and 6B illustrate that FcγRIIB expression is required for reversal of immune thrombocytopenia by soluble OVA in the presence of anti-OVA.

FIGS. 7A and 7B illustrate that FcγRIIB expression is not required for reversal of immune thrombocytopenia by cell-associated OVA in the presence of anti-OVA.

FIGS. 8A and 8B illustrate that antibodies to endogenous soluble antigens ameliorate immune thrombocytopenia.

FIG. 9 illustrates that antibodies to albumin and transferrin require the expression of FcγRIIB to ameliorate immune thrombocytopenia.

FIGS. 10 A and 10B illustrate that antibodies to albumin ameliorate K/B×N serum-induced inflammatory arthritis.

FIG. 11 illustrates IMCP-like effects shown by IVIg and anti-CD44 treatment regimes.

FIG. 12 illustrates IMCP-like effects as shown by IVIg and soluble antigen-specific antibody treatment regimes.

FIG. 13 illustrates IVIg-treated leukocytes showing therapeutic potential in the absence of FCgammaRIIB expression.

### DETAILED DESCRIPTION OF THE PREFERRED EMBODIMENT

In this description by soluble antigen it is meant a molecule that can be incorporated and circulated in the blood stream. Examples of soluble antigens comprise but are not limited to: proteins, glycoproteins, lipids, glycolipids, peptides, nucleic acids, synthetic molecules or complexes or aggregates thereof.

By endogenous antigen it is meant antigens that occur naturally in a mammal and by foreign (or exogenous) antigen it is meant an antigen that is not normally produced by the same individual or species.

According to one embodiment of the present invention, antibodies to soluble antigens were tested for their ability to ameliorate autoimmune diseases. In one example, the amelioration of thrombocytopenia was tested. To address this question, a murine model of ITP was used to determine whether IgG specific to a soluble prototype antigen could prevent thrombocytopenia. Mice injected with soluble ovalbumin (OVA) or OVA conjugated to RBCs (OVA-RBC) in the presence of anti-OVA, were both significantly protected from immune thrombocytopenia.

Both of these therapeutic regimes functioned independent of complement activity and both regimes also blocked reticuloendothelial function as assessed by clearance rates of fluorescent sensitized syngeneic RBCs. Soluble OVA or anti-OVA alone did not have any direct effect on immune thrombocytopenia in mice. It was found that OVA-RBC+anti-OVA ameliorated immune thrombocytopenia in normal mice, not in FcγRIIB^{-/-} mice, while soluble OVA+anti-OVA was ineffective. In addition, IgG specific for murine albumin and specific for transferrin also effectively inhibited ITP. Thus, IgG antibodies directed to soluble antigens can inhibit or reverse immune thrombocytopenia in an FcγRIIB-dependent manner, whereas antibodies directed to a cell-associated antigen function independent of FcγRIIB expression.

### Materials and Methods:

### Reagents:

The monoclonal antibody specific for integrin α_{IIb} (rat IgG₁ₖ, clone MWReg 30) was purchased from BD Pharmigen (Mississauga, ON, Canada). Monoclonal murine anti-OVA (IgG₁, clone OVA-14), rabbit polyclonal anti-OVA, 1-ethyl-3-(3-dimethylamino-propyl)carbodiimide (EDAC), OVA (grade V), and PKH26 red fluorescent cell linker kit were purchased from Sigma (Oakville, ON, Canada). IVIG was Gamimune, 10% from Bayer (Elkhart, Ind.). Cobra Venom Factor (CVF), FITC-conjugated F(ab′)₂ anti-rabbit IgG, and control rabbit IgG, were purchased from Cedarlane Laboratories Ltd (Hornby, ON, Canada). Rabbit anti-mouse albumin (IgG fraction), and rabbit anti-mouse transferrin (IgG fraction), were purchased from Research Diagnostics (Flanders, N.J.). Hemolysin (anti-SRBC rabbit serum) was supplied by Colorado Serum company (Denver, Colo.). Microdispenser tubes (250 μl) for blood collection were from VWR, (Mississauga, ON)

### Mice:

Female CD1 mice (6-10 wks of age) were purchased from Charles River Laboratories (Montreal, PQ, Canada). C57BL/6 and FcγRIIB^{-/-} (B6;129S-Fcgr2^{tm1Rav}/J) mice were purchased from the Jackson Laboratory (Bar Harbor, Me.). All mice were housed in the St. Michael's Hospital Research Vivarium.

### Induction and Treatment of Immune Thrombocytopenia:

Mice were rendered thrombocytopenic by intraperitoneal injection of 2 μg anti-platelet (anti-integrin α_{IIb}) antibody in 200 μl phosphate buffered saline (PBS), pH 7.2. ITP was induced by two protocols:

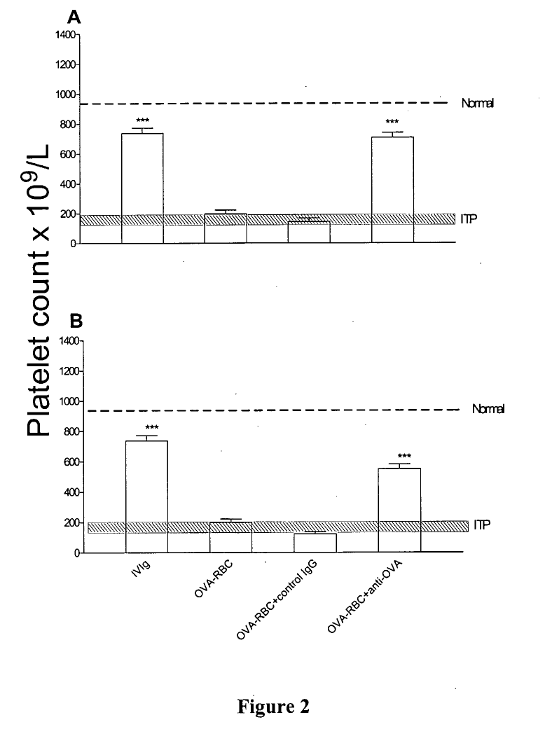

For experiments where the therapeutic intervention preceded the induction of immune thrombocytopenia (e.g. FIGS. 2, 3, 5), mice were first injected intravenously with the indicated therapeutic preparation (eg OVA-RBC sensitized with anti-OVA IgG), followed at 24 h by a single injection of anti-platelet antibody. Mice were bled for platelet enumeration after a further 24 h.

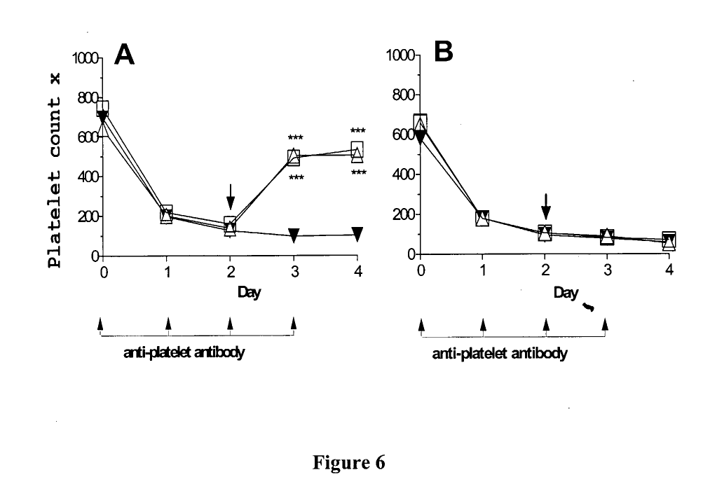

For experiments where the induction of immune thrombocytopenia preceded the therapeutic intervention (e.g. FIGS. 6-8), mice were injected daily (days 0-3) with anti-platelet antibody and then injected intravenously with the indicated therapeutic preparation (eg OVA-RBC sensitized with anti-OVA IgG) on day 2. Mice were bled daily and platelets counted as described below.

In experiments where we wished to avoid the possibility of the formation of “pre-formed” immune complexes, mice were injected intraperitoneally with soluble OVA only followed 4 hours later by OVA-specific antibody via the intravenous route. Mice injected with anti-albumin or anti-transferrin alone received 1 mg of antibody in a volume of 200 ul on day 2. For all IVIg treatments, mice were injected intraperitoneally with 0.5 ml of 10% IVIG (roughly equivalent to 2 g/kg body weight) . Platelets were counted as follows: Mouse blood (45 μL) was collected via saphenous vein bleeding into microdispenser tubes preloaded with 5 μl of 1% EDTA in PBS. Then, 50 μl of this mouse blood was diluted in 450 μl of 1% EDTA/PBS (1:10) and then further diluted to a final dilution of 1:12,000 in 1% ethylenediaminetetraacetic acid (EDTA)/PBS. Platelets were enumerated on a flow rate-calibrated FACScan flow cytometer (Becton Dickinson, San Jose, Calif.) using forward scatter (FCS) versus side scatter (SSC) to gate platelets as previously described.

### Preparation of OVA-Specific Antibody Pre-Incubated with Soluble OVA:

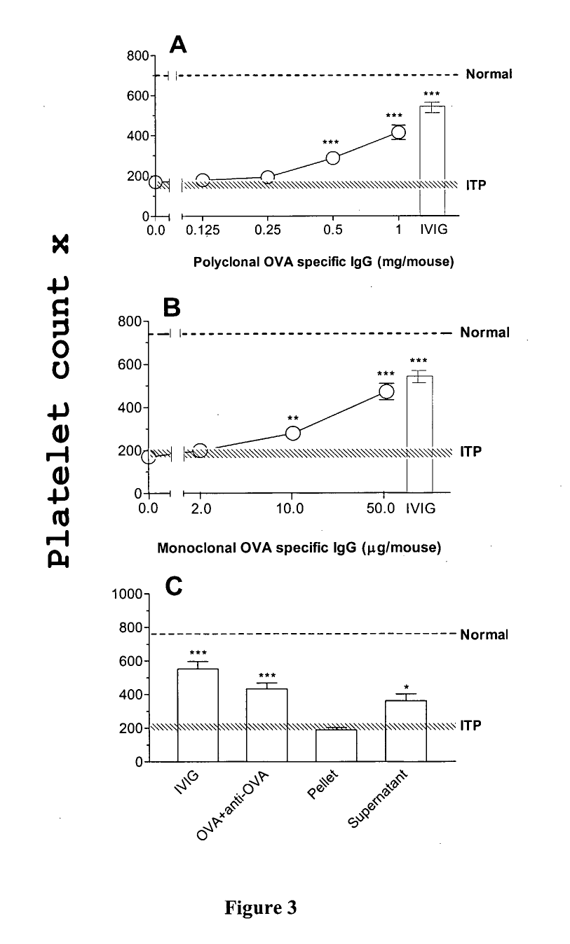

1 mg OVA was dissolved in 300 μl PBS and was incubated with the indicated dose (FIG. 3A, 3B x-axes) of OVA-specific antibody (rabbit polyclonal or mouse monoclonal) for 1 hr at 37° C. The solution was then injected intravenously in a 300 μ1 volume. In separate experiments the OVA and antibody solution was incubated as above for 1 hour at 37° C. and macromolecular immune complexes removed by centrifugation at 16,000×g at 4° C. for 1 h followed by filtration of the resulting supernatant fluid using a 0.2 μm filter (Filtropur S plus 0.2, Sarstedt, Montreal, PQ). The pellet was resuspended in 300 μl PBS and intravenously injected as above.

### Preparation of OVA-Coupled RBCs:

OVA was coupled to RBCs as follows: RBCs were resuspended at 2.5×10⁸/mL in 5 mg/mL OVA in saline and 1.9 mg/mL 1-ethyl-3-(3-dimethylamino-propyl) carbodiimide (EDAC) was added. Following a 1 hr incubation at 4° C., the cells were washed once with a 2 mg/mL solution of OVA in PBS followed by one wash in PBS. To confirm the presence of OVA on RBCs, OVA coupled RBCs were incubated with 17 μg/mL rabbit polyclonal anti-OVA, washed, and then incubated with 8 μg/mL FITC conjugated F(ab′)₂ anti-rabbit IgG. Cells were washed, resuspended in PBS, and analyzed by flow cytometry.

### Reticuloendothelial System (RES) Blockade:

RES blockade was assessed as follows: Whole blood (2 ml, diluted with 1/5 volume 1% EDTA in PBS) from unmanipulated SCID mice was pooled and centrifuged at 2,000×g for 15 min to obtain 1 ml of packed erythrocytes. These packed erythrocytes were resuspended in 4 ml of PBS and incubated with 8 μg of anti-TER-119 antibody at 22° C. for 30 min. The resulting opsonized erythrocytes were then washed twice with PBS and labeled with a fluorescent marker (PKH26 Kit, Sigma, St. Louis Mo.) according to the manufacturer's directions. Briefly, the opsonized erythrocytes were resuspended in 3 ml of PKH26 ‘diluent C’ and mixed with another 4 ml of ‘diluent C’ containing 10 μl of the ‘PKH26 linker’. After a 5 minute incubation with constant swirling, the mixture was incubated for 5 minutes with an equal volume of PBS containing 1% bovine serum albumin. The erythrocytes were washed 5 times and resuspended in 2 ml PBS. Mice were then injected via the tail vein with 200 μl of these labeled cells. All mice were bled via the tail vein at 3 min, 10 min, 30 min, 120 min, and 960 min post injection and the the total number of erythrocytes, as well as the percent of PKH26-fluorescent erythrocytes, were counted by flow cytometry. The percentage of labeled erythrocytes at the 3 min time point was considered to be 100%.

### Complement Depletion:

Complement depleted mice were prepared by intraperitoneal injection of 5 U of Cobra Venom Factor (CVF) in 200 μl phosphate-buffered saline pH 7.2 followed by a second injection of CVF after 4 h. Complement depletion was confirmed by the complement hemolytic activity assay Briefly, sheep RBCs (SRBC) were washed in PBS and resuspended at 1×10⁸/mL. Hemolysin (anti-SRBC rabbit serum) was diluted 1:50 and incubated with these sheep RBCs at 37° C. for 30 min, washed in PBS and the cells incubated with a 1:10 dilution of mouse sera from control vs. CVF-treated mice at 37° C. for 30 min. The mixture was then diluted with PBS, centrifuged at 1000×g for 5 min. Complement activity from the sera was assessed as follows: SRBC were resuspended in PBS at 1×10⁸/mL. One mL of this was incubated with 1 mL of a 1/50 dilution of anti-SRBC antibody (‘Hemolysin’, Colorado serum, Denver, Colo.) and incubated for 30 min at 37° C. Cells were washed in PBS, and adjusted to 1×10⁸/mL in PBS. Twenty mL of these cells were added to 20 μl mouse serum from experimental mice in a 96 well flat bottom tissue culture plate for 30 min at 37° C. The plate was then centrifuged at 1,000×g for 5 min, the supernatant was transferred to a new 96 well plate and the absorbance was read at 540 nm. Calculate percent hemolysis: 100×(OD₅₄₀sample−OD₅₄₀blank)/(OD₅₄₀max−OD₅₄₀blank). Calculate 50% lysis by plotting the log of serum dilution against log (% lysis/(100−% lysis)).

### Statistical Analysis:

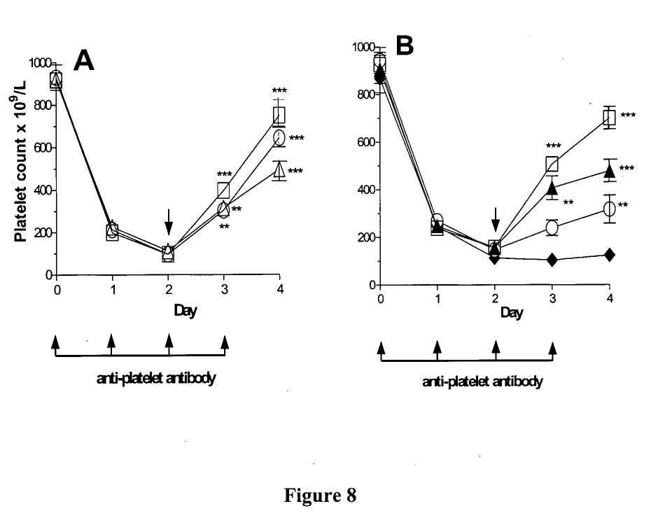

Data was analyzed using the Student's t test, except data in FIG. 8, which was analyzed by one-way ANOVA. The level of significance was set at P<0.05.

### Results

### Antibodies Reactive with a Cell-Associated Antigen can Inhibit Immune Thrombocytopenia:

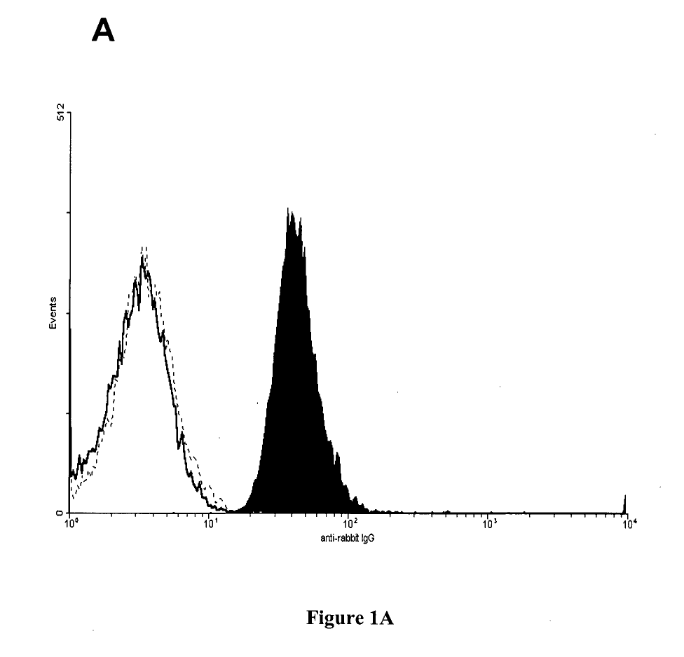

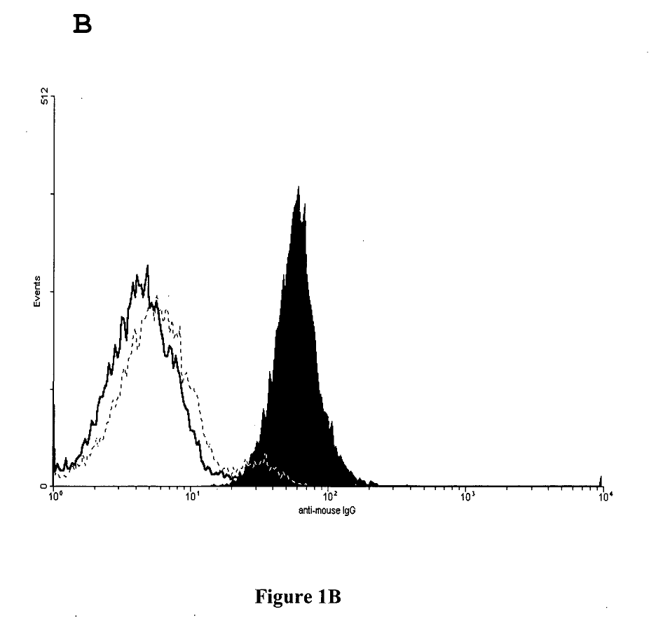

OVA-coupled murine RBCs (OVA-RBC) were assessed for reactivity with mouse (FIG. 1A) and rabbit (FIG. 1B) antibody specific to OVA by flow cytometry to ensure successful coupling of the OVA-RBCs. FIGS. 1A and 1B illustrate the association of OVA on the surface of RBCs wherein OVA coupled RBCs are prepared with 1-ethyl-3-(3-dimethylamino-propyl) carbodiimide (EDAC) (Sigma Oakville, ON). OVA was coupled to RBCs as follows: RBCs were resuspended at 2.5×108/mL in 5 mg/mL OVA in saline and 1.9 mg/mL EDAC was added. Following a 1 hr incubation at 4° C., the cells were washed once with a 2 mg/mL solution of OVA in phosphate buffered saline (PBS), pH 7.2 followed by one wash in PBS. The OVA coupled RBCs were stained with rabbit (FIG. 1A) or mouse (FIG. 1B) polyclonal anti-OVA IgG (solid histogram), control rabbit (FIG. 1A) or mouse (FIG. 1B) IgG (solid line), followed by the appropriate FITC conjugated secondary antibody (dashed line, secondary antibody only) and wherein the x axis shows relative fluorescence intensity; y-axis represents cell number.

The monoclonal anti-OVA antibody employed in this study did react with OVA (as assessed by ELISA), but did not react with OVA-RBCs suggesting that the epitope recognized on OVA may be masked upon coupling with RBCs. Thus monoclonal anti-OVA was only used in treatments involving soluble OVA.

CD1 mice were injected intravenously with 1×10⁸ OVA-RBCs pre-incubated with nothing, OVA specific antibodies or an appropriate control IgG, 50 mg IVIg (roughly equivalent to 2 g/kg body weight in a 25 g mouse), or were left untreated. After 24 hours, all mice received anti-platelet antibody and all mice were bled for platelet enumeration after a further 24 h. Mice that received anti-platelet antibody alone became thrombocytopenic (FIG. 2, shaded horizontal bar), compared to unmanipulated control mice (FIG. 2, dashed line). FIGS. 2A and 2B illustrates inhibition of thrombocytopenia by treating OVA-coupled RBCs with OVA-specific IgG; CD1 mice were pre-injected intravenously with 1×10⁸ OVA-coupled RBCs pre-incubated with either rabbit (A) or mouse (B) OVA-specific polyclonal IgG, control IgG, or anti-OVA antibody, as indicated on the x axis. Mice in the IVIG groups received 50 mg IVIG. All mice (except ‘Normal’) received anti-platelet antibody one day later. Mice were bled for platelet enumeration after a further 24 h. Normal: The dashed line denotes the mean platelet count of non-injected mice; ITP: The horizontal bar denotes the mean platelet count (±1 SEM) of mice injected with anti-platelet antibody only. The x-axis indicates treatment; y-axis denotes platelet count; n=9 mice for each data point. ***P<0.001 vs. ITP mice. Data are represented as mean ±.SEM.

Mice treated with OVA-RBCs pre-incubated with either 50 μg rabbit polyclonal anti-OVA (FIG. 2A, ‘OVA-RBC+anti-OVA’) or 50 μg murine polyclonal anti-OVA (FIG. 2B, ‘OVA-RBC+anti-OVA’) were significantly protected from the development of immune thrombocytopenia compared with mice receiving OVA-RBCs alone (OVA-RBC) or OVA-RBC+control IgG (OVA-RBC+control IgG). The effectiveness of the IgG coated OVA-RBCs was comparable to that of IVIg (FIGS. 2A&B).

### Antibodies Reactive with a Soluble Antigen can Inhibit Immune Thrombocytopenia:

CD1 mice were injected intravenously with 1 mg soluble OVA that had been pre-incubated with serial dilutions of the indicated amount of rabbit polyclonal anti-OVA (FIG. 3A) or the indicated amount of murine monoclonal anti-OVA antibody (FIG. 3B) one day prior to injection of anti-platelet antibody. Both of these therapeutic preparations ameliorated immune thrombocytopenia (polyclonal anti-OVA at dosages of 1.0 or 0.5 mg/mouse, monoclonal at dosages of 50 or 10 ug/mouse).

CD1 mice were pre-injected intravenously with 1 mg OVA pre-incubated with the dose of rabbit polyclonal anti-OVA (A), or mouse monoclonal anti-OVA (B), as indicated on the x axis. Mice in the IVIG groups received 50 mg IVIG. The induction of thrombocytopenia and platelet counting were as in FIG. 2. Panel C: the OVA/polyclonal anti-OVA solution was centrifuged and the supernatant fluid filtered using a 0.2 um filter to remove macromolecular immune complexes. The pellet was resuspended in PBS. Mice were injected with the therapeutic preparations indicated on the x axis. The induction of thrombocytopenia, platelet counting, and axis legends are as in FIG. 2. The number of mice for data point were n=15 (A, B), n=4 (C). ***P<0.001 vs. ITP mice. Data are represented as mean±SEM.

It is of interest to note that OVA incubated with 50 ug monoclonal anti-OVA was essentially as successful at inhibiting ITP as was a standard dose of IVIg (FIG. 3B). Mice treated with soluble OVA alone (FIGS. 3A&B, 0.0 mg/mouse) or OVA+control IgG (data not shown) were not significantly protected from the development of immune thrombocytopenia. OVA by itself did not affect the platelet count at any dose tested (0.1 mg, 1 mg, 5 mg and 10 mg, data not shown). Similarly all of the anti-OVA antibodies, in the absence of OVA, did not inhibit immune thrombocytopenia (data not shown).

To determine if the OVA+anti-OVA preparation ameliorated immune thrombocytopenia due to the formation of large macromolecular immune complexes, we subjected the OVA+polyclonal anti-OVA preparation (1 mg:1 mg) to centrifugation at 16,800×g for 1 hr. at 4° C. and the resulting supernatant was then filtered through a 0.2 uM filter (Filtropur S plus 0.2, Sarstedt, Montreal, PQ). Pretreatment of mice with the filtered supernatant, but not the dissolved pellet(the pellet was dissolved by resuspending the pellet in PBS, pH 7.2, back to the original volume), prior to injection of anti-platelet antibody protected mice from thrombocytopenia (FIG. 3C), suggesting that the “active” fraction was soluble and less than 0.2 uM in size.

### Antibodies Reactive with Soluble and a Cell-Associated Soluble Antigen both Block RES Function:

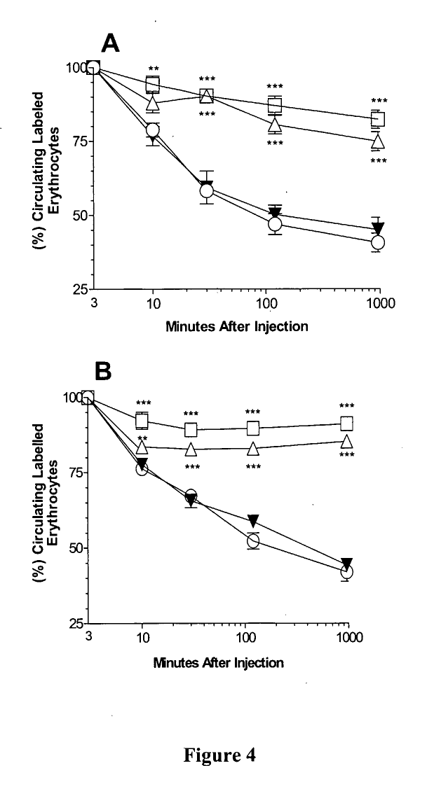

To assess whether the therapeutic regimes under study inhibited RES function, we employed a variation of the classic RES blockade assay, analysing the clearance rate of fluorescently labelled syngeneic RBCs sensitised with a murine RBC-specific antibody (anti-TER-119). Mice were subjected to the indicated therapeutic treatments, and their ability to clear these intravenously injected labelled RBCs over time was analysed (FIG. 4). For the soluble antigen studies, mice were injected with nothing, IVIg, OVA-anti-OVA, or control IgG alone for 24 h followed by sensitized fluorescent RBCs (FIG. 4A). At the indicated times post sensitized-fluorescent-RBC injection, blood was sampled to assess the RBC clearance rate as a measure of RES function. Only IVIg and OVA-anti-OVA blocked sensitized RBC clearance. Similar results were obtained with murine anti-OVA in combination with soluble OVA (data not shown).

For the cell-associated antigen studies, mice were injected with nothing, IVIg, anti-OVA sensitized OVA-RBCs, or OVA-RBCs alone for 24 h followed by sensitized fluorescent RBCs (FIG. 4B). Only IVIg and anti-OVA sensitized OVA-RBCs blocked sensitized-fluorescent-RBC clearance.

In accordance with FIGS. 4A and 4B, mice were either not pre-treated (∘), pre-treated with IVIG (□), pre-treated with 1 mg OVA pre-incubated with 1 mg rabbit anti-OVA (Δ), or pre-treated with 1 mg control IgG+1 mg OVA (▾), followed 24 hours later by intravenous injection with fluorescently labeled TER-119-opsonized syngeneic RBCs, prepared as follows: Whole blood (2 ml, diluted with 1/5 volume 1% EDTA in PBS) from unmanipulated mice was pooled and centrifuged at 2,000×g for 15 min to obtain 1 ml of packed erythrocytes. These packed erythrocytes were resuspended in 4 ml of PBS and incubated with 8 μg of anti-TER-119 antibody at 22° C. for 30 min. The resulting opsonized erythrocytes were then washed twice with PBS and labeled with a fluorescent marker (PKH26 Kit, Sigma, St. Louis Mo.) as follows: Briefly, the opsonized erythrocytes were resuspended in 3 ml of PKH26 ‘diluent C’ and mixed with another 4 ml of ‘diluent C’ containing 10 μl of the ‘PKH26 linker’ . After a 5 minute incubation with constant swirling, the mixture was incubated for 5 minutes with an equal volume of PBS containing 1% bovine serum albumin. The erythrocytes were washed 5 times and resuspended in 2 ml PBS. Mice were then injected via the tail vein with 200 μl of these labeled cells. All mice were bled via the tail vein at 3 min, 10 min, 30 min, 120 min, and 960 min post injection and the total number of erythrocytes, as well as the percent of PKH26-fluorescent erythrocytes, were counted by flow cytometry. The percentage of labeled erythrocytes at the 3 min time point was considered to be 100%.

Blood samples were taken at the times indicated on the x axis and the percentage of fluorescent RBCs in the circulation assessed by flow cytometry (FIG. 4B), mice were either not pre-treated (∘), pre-treated with IVIG (□), pre-treated with anti-OVA sensitized OVA-RBCs (Δ), or pre-treated with OVA-RBCs only (▾) followed 24 hours later with intravenous injection of fluorescently labelled TER-119-opsonized autologous RBCs.

### Antibodies Reactive with Soluble or Cell-Associated Soluble Antigen Inhibit ITP-Independent of Complement Activity:

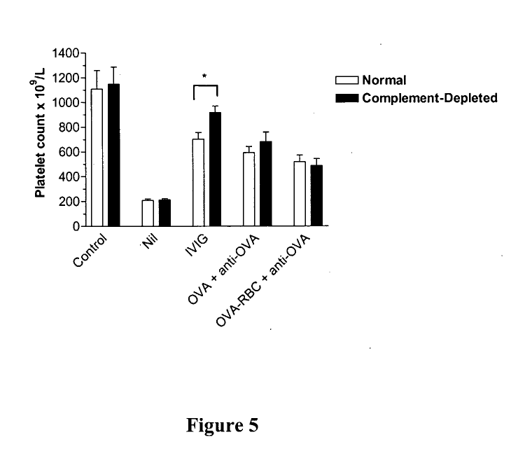

To determine if complement was a contributing factor to the above therapies, mice were depleted of Complement using cobra factor venom (CVF) as described above in [46]._CVF successfully depleted complement from the treated mice as assessed in a hemolytic activity assay on day 3 post CVF-treatment (data not shown). Complement depleted mice developed thrombocytopenia to the same extent as normal mice (FIG. 5, column set 2). Complement depleted and normal mice both responded to the protective effects of OVA+anti-OVA and OVA-RBC+anti-OVA (column sets 4 and 5 respectively) to the same extent. However, complement depleted mice responded to IVIg treatment with significantly higher platelet counts compared with normal mice.

As shown in FIG. 5, antibodies reactive with soluble OVA or OVA-RBCs both ameliorate immune thrombocytopenia independent of complement activity wherein mice were injected with CVF to deplete complement or were left untreated. After 24 hours, mice were treated with the therapeutic preparations indicated on the x axis, the induction of thrombocytopenia and platelet counting were as in FIG. 2, control: mice receiving no therapeutic pre-treatment; Nil: mice treated with anti-platelet antibody only; ‘OVA+anti-OVA’: mice pre-treated with OVA+anti-OVA, followed 24 hr later by injection of anti-platelet antibody. ‘OVA-RBC+anti-OVA’ : mice pre-treated with OVA-RBC+anti-OVA, followed 24 hr later by injection of anti-platelet antibody.

### FcγRIIB Expression is Required for Protection with Antibodies Reactive with Soluble, but not a Cell-Associated Antigen:

Wild type and FcγRIIB^{-/-} mice were injected daily with anti-platelet antibody (↑) to induce stable thrombocytopenia (FIG. 6). Mice were then treated with IVIg, OVA+anti-OVA, or control IgG+OVA on day 2. Treatment of mice with 2 g/kg IVIg as well as OVA+anti-OVA successfully reversed immune thrombocytopenia in wild type (FIG. 6A), but neither ameliorated ITP in FcγRIIB^{-/-} mice (FIG. 6B). Mice treated with control IgG+OVA displayed no increase in platelet counts.

FIGS. 6A and 6B illustrate that FcγRIIB expression is required for reversal of immune thrombocytopenia by soluble OVA in the presence of anti-OVA wherein wild type mice (FIG. 6A) or mice genetically deficient for FcγRIIB (FcγRIIB−/−) mice (FIG. 6B) were injected with anti-platelet antibody on days 0 through 3 denoted by the arrow (↑), on day 2 (↓) mice were injected intraperitoneally with IVIG (□), or intravenously with OVA+anti-OVA antibody (Δ), or non-specific IgG+OVA (▾) and mice were bled daily for platelet counts (×10⁹/L).

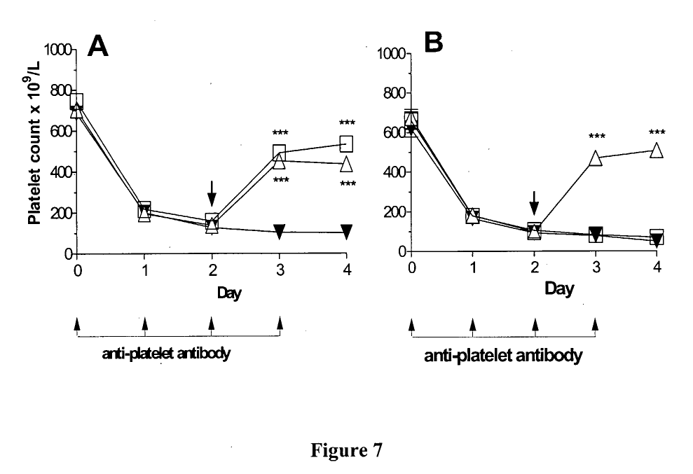

In sharp contrast to the results in FIG. 6, ITP was successfully reversed in normal mice (FIG. 7A) and FcγRIIB^{-/-} mice (FIG. 7B) that were therapeutically treated with OVA-RBCs+anti-OVA. As expected, treatment of mice with OVA-RBCs alone did not increase platelet counts in thrombocytopenic mice. FIGS. 7A and 7B illustrate that FcγRIIB expression is not required for reversal of immune thrombocytopenia by cell-associated OVA in the presence of anti-OVA wherein wild type mice (FIG. 7A) or FcγRIIB^{-/-} mice (FIG. 7B) were injected with anti-platelet antibody on days 0 through 3 (↑), on day 2 (↓) mice were injected intraperitoneally with IVIG (□), or intravenously with anti-OVA sensitized OVA-RBCs (Δ), or OVA-RBCs alone.

### Preformation of Immune Complexes are not Necessary for Reversal of ITP:

To determine if it is necessary to incubate antigen and antibody before injection to ameliorate the thrombocytopenia in our model, mice were pre-injected with either 1 mg or 10 mg of soluble OVA followed by 1 mg anti-OVA after 4 h. Significant reversal of ITP was achieved with OVA specific IgG in mice that were previously treated with either 1 mg or 10 mg of OVA (FIG. 8A).

To determine if antibody to endogenous soluble antigens can also inhibit immune thrombocytopenia, thrombocytopenic mice were treated with 1 mg polyclonal anti-mouse albumin or 1 mg anti-mouse transferrin antibody on day 2. Both of these antibodies, but not control IgG, significantly ameliorated the immune thrombocytopenia (FIG. 8B). As illustrated in FIGS. 8A and 8B, antibodies to endogenous soluble antigens ameliorate immune thrombocytopenia wherein (FIG. 8A) mice were treated with IVIG only (□), 10 mg OVA (Δ), or 1 mg OVA (∘), followed four hours later by 1 mg OVA-specific IgG (↓) on day 2 and wherein thrombocytopenia and platelet counting were as in FIG. 6 and wherein (FIG. 8B) mice were treated with IVIG (□), 1 mg anti-mouse albumin antibody (▴), 1 mg anti-mouse transferrin antibody (∘), or control IgG (♦).

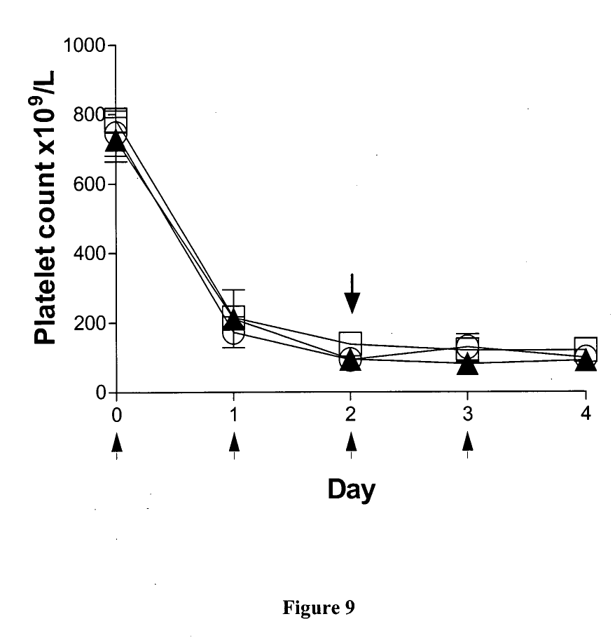

In contrast, anti-mouse albumin and anti-mouse transferrin antibodies failed therapeutically in FcγRIIB^{-/-} mice, and did not reverse immune thrombocytopenia (FIG. 9). Here, antibodies to albumin and transferrin require the expression of FcγRIIB to ameliorate immune thrombocytopenia. FcγRIIB^{-/-} mice were injected with 2 μg anti-platelet antibody on days 0 through 3 denoted by the arrow (↑). On day 2 (↓) mice were injected intraperitoneally with 50 mg IVIg (□), or intravenously with 1 mg anti-albumin antibody (▴), or 1 mg anti-transferrin antibody (∘). Mice were bled daily for platelet counting; n=3 mice for each group. Data are presented as mean±SEM.

In another embodiment of the invention antibodies to soluble antigens were used to treat or ameliorate inflammatory arthritis.

### Material and Methods

### K/B×N Serum-Induced Arthritis and Arthritis Scoring:

For induction of arthritis, mice were given a single intraperitoneal injection of 600 μl of diluted serum (diluted to 50% strength with PBS) as previously described by Akilesh et al (Akilesh, S., Petkova, S., Sproule, T. J., Shaffer, D. J., Christianson, G. J., and Roopenian, D. 2004. The MHC class I-like Fc receptor promotes humorally mediated autoimmune disease. J Clin Invest 113:1328-1333.). An additional control group of mice were injected with only PBS instead of K/B×N serum. Ankle width was measured laterally across the joint with a caliper (Samona International, Canada). Arthritis was also clinically scored daily by an independent blinded observer. Each paw was scored as follows: 0, [unaffected], 1 [slight swelling], 2 [moderate swelling], 3 [severe swelling involving the entire paw (foot, digits, ankle)], and the overall score was calculated as the sum of individual scores for each paw as described by de Fougerolles et al (de Fougerolles, A. R., Sprague, A. G., Nickerson-Nutter, C. L., Chi-Rosso, G., Rennert, P. D., Gardner, H., Gotwals, P. J., Lobb, R. R., and Koteliansky, V. E. 2000. Regulation of inflammation by collagen-binding integrins alpha1beta1 and alpha2beta1 in models of hypersensitivity and arthritis. J Clin Invest 105:721-729.). Mice injected with anti-albumin or the IgG control received 1 mg of IgG intravenously in 200 μl PBS four hours prior to the induction of arthritis. Mice injected with IVIg received 50 mg of IVIg by an intraperitoneal injection four hours prior to the induction of arthritis.

### IgG Reactive with a Soluble Antigen can Ameliorate Arthritis:

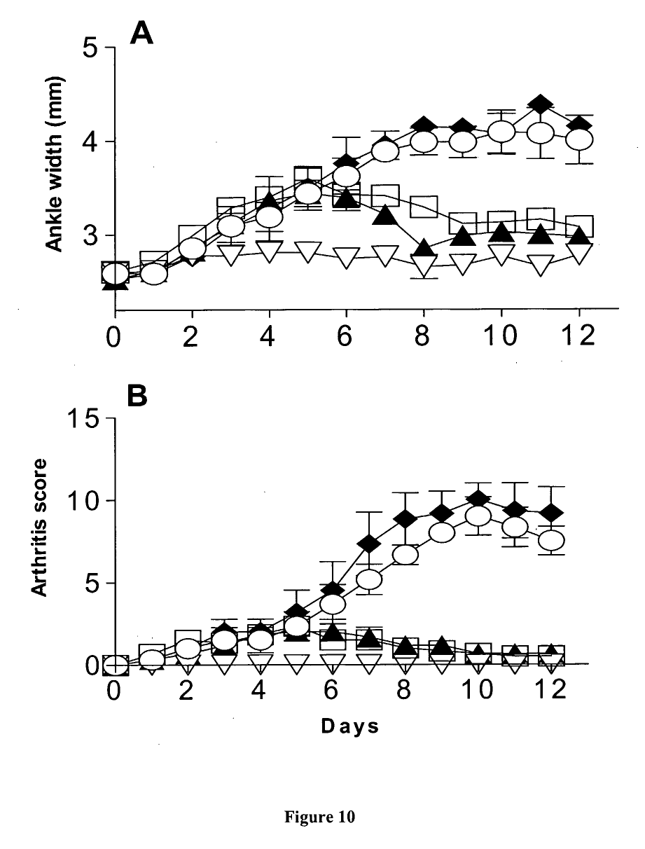

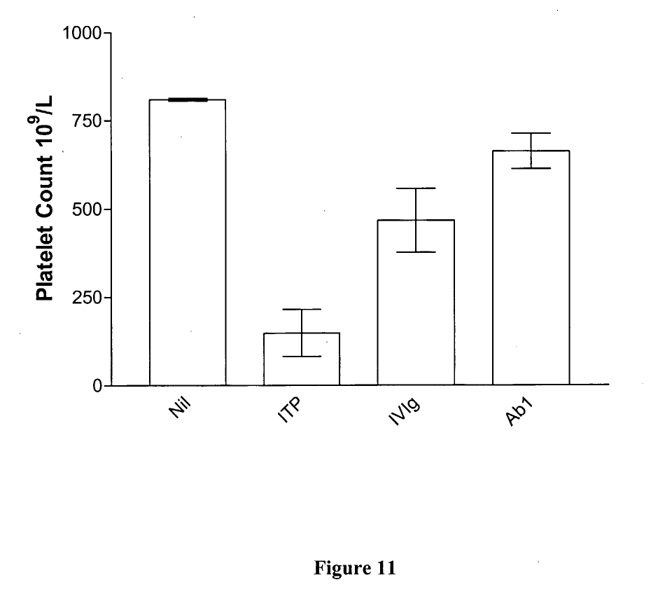

To further evaluate the therapeutic role of antibodies directed to a soluble antigens in the K/B×N serum-induced arthritis model, C57BL/6 mice were injected with 50 mg IVIg, 1 mg anti-albumin, 1 mg non-immune IgG, or nothing 4 hours prior to receiving K/B×N serum. An additional control group of mice were injected with only PBS in place of the K/B×N serum. Mice that received K/B×N serum alone, or K/B×N serum+non-immune IgG, developed joint swelling (FIGS. 10A and B). As shown in FIGS. 10A & B, antibodies to albumin ameliorate K/B×N serum-induced inflammatory arthritis. (A) Ankle width and (B) overall arthritis score following K/B×N serum-induced arthritis. C57BL/6 mice were injected on day 0 with K/B×N serum (∘), IVIg+K/B×N serum (□), anti-albumin+K/B×N serum (▴), Non-immune IgG+K/B×N serum (♦), or treated with only PBS in place of K/B×N serum (∇). Date represented as the mean±SEM; n=3 mice for each group.

IVIg and the anti-albumin treatment significantly ameliorated the arthritis as assessed by ankle width measurements as well as by clinical score as compared to mice that received K/B×N serum or K/B×N serum plus treatment with non-immune IgG (FIGS. 10A and B).

### Mechanism of Action:

Our further investigation has also revealed surprising evidence for the mechanism of action of the treatment regimes as herein disclosed. In particular, we have established that antibody treatment regimes such as IVIg, a monoclonal antibody to CD44 antigen and anti-soluble immune complex antibodies (in the presence of the antigen) work to ameliorate autoimmune disease via an antibody-mediated cellular programming mechanism, otherwise herein referred to as IMCP, of non-B and non-T cell leukocytes. In particular, we show that IVIg, monoclonal antibody to CD44 antigen and anti-soluble immune complex antibodies (in the presence of the antigen) can bind to leukocytes in vitro and upon transfer in vivo, can ameliorate ITP, for example. More specifically, IVIg, a monoclonal antibody to the CD44 antigen, and anti-soluble immune complex antibodies (in the presence of the antigen) ameliorate autoimmune disease by interacting with a non-B cell non-T cell leukocyte which then, upon transfer to a host with an autoimmune disease, ameliorates disease activity. We have found that the leukocyte which mediates these clinical effects co-purifies with cells, including a subset of intestinal epithelial lymphocytes and a subset of activated T-cells, expressing the CD11c cell surface antigen, a surface marker expressed on most dendritic cells [data not shown]. Thus, a novel mechanism of action for IVIg and IVIg-like treatment regimes for autoimmune disease is herein provided.

Furthermore, a common linking factor is established in that the expression of FCgamma RIIB inihibitory receptor on cells is shown in the treatment regimes for anti-CD44 and antibodies directed to a soluble antigens, as has been previously established for IVIg. Thus, providing evidence that a common mode of action is the basis for the treatment regimes of the present invention. Having established a common mechanism of action with IVIg, anti-CD44 antibody, we believe that an antibody for a soluble antigen, in accordance with the present invention, will have a similar therapeutic effect as IVIg or anti-CD44 antibody, in the treatment and/or amelioration of a plurality of autoimmune diseases. Accordingly, the embodiments of the present invention may be extended to provide beneficial treatment regimes for the prevention and/or treatment of other autoimmune diseases.

### Materials and Methods

### Mice:

CD1 mice (female 6-10 wk of age) and severe combined immune deficient (SCID) virgin mice (female 6 to 8 weeks of age) were purchased from Charles River Laboratories (Montreal, PQ, Canada). C57BL/6, BALB/c, and FcγRIIB^{-/-} mice were (female 8 to 12 weeks of age) were from the Jackson Laboratory (Bar Harbor, Me.).

### Reagents:

The monoclonal antibody specific for integrin αIIb (rat IgG₁ₖ, clone MWReg 30) was purchased from BD Pharmingen (Mississauga, ON). Bovine serum albumin (BSA) was purchased from Sigma (Oakville, ON, Canada). The IVIg (Gamimune N, 10%) was from Bayer (Elkhart, Ind.). To neutralize the pH of the IVIg (in some experiments), both IVIg and BSA were dialysed against phosphate buffered saline (PBS) (pH 7.2) in 1:200 ratio for 18 hours at 4° C. using 12-14 kDa cutoff dialysis tubing (Spectrum Laboratories Inc, Rancho Dominguez, Calif.) under sterile conditions. Microdispenser tubes (250 μL) for blood collection were from VWR. Complete RPMI-1640 was RPMI-1640 medium (Sigma, Oakville, ON, Canada) supplemented with 10% heat-inactivated fetal calf serum, 80 μg/ml streptomycin sulphate, 0.2 μg/ml amphotericin B, 80 U/ml penicillin G and 1.6 mM L-glutamine.

### IVIg-Mediated Cellular Programming (IMCP):

### Preparation of IMCP Blood:

Blood (400 μl, or as otherwise indicated) was collected in sterile PBS containing 1% EDTA (PBS/EDTA), washed and the cell pellets resuspended in 25 mg/ml of IVIg or BSA in PBS/EDTA. After incubation for 20 min (or as otherwise indicated) at 37° C. in a shaking incubator, the cells were washed 2× in Ca⁺⁺ and Mg⁺⁺ free PBS, resuspended in saline and immediately injected back into the original mice. For preparation of WBC-reduced blood cells, the collected blood was first centrifuged at 900×g for 5 min at 4° C., the plasma and buffy coat fractions were discarded. The cell pellets were washed 3× in PBS and resuspended in 25 mg/ml of IVIg or BSA as described above.

### Preparation of IMCP Splenic Cells:

Spleens from normal mice were removed, mechanically disrupted in 5 ml of complete RPMI-1640 medium, and then filtered through 70-μm nylon mesh strainer. Erythrocytes were lysed using 0.15 M NH4Cl, 10 mM KHC03, 0.1 mM Na2 EDTA (ACK) lysis buffer and washed 2× in RPMI-1640. The cells (1.4×106/ml) were incubated with 18 mg/ml dialyzed IVIg (IMCP) or BSA (IMCP-control), or the indicated concentration (x/ml) of anti-CD44 (Antibody clone KM-114 or IM7), or with 1 mg of ovalbumin that was pretreated with 50 ug monoclonal anti-ovalbumin (Clone OVA-14, antibody subclass IgG1, From Sigma), or 1 mg of ovalbumin that was pretreated with 50 ug normal mouse IgG (Catalogue # 10400, from Caltag) for 30 min at 370° C. in RPMI-1640. The cells were then washed 2× in RPMI-1640, resuspended to 5×106/ml and injected (200 μl) into the tail vein of recipient mice.

### Fixation:

Pre-fixed cells: splenic leukocytes (2.5×106/ml) were fixed in 1% paraformaldehyde in PBS for 10 minutes, washed 2× in PBS and then incubated with IVIg or BSA for 30 min as described above.

Post-fixed cells: splenic leukocytes were first incubated with IVIg or BSA for 30 min as described above, washed 2× in PBS and then fixed in 1% paraformaldehyde in PBS. The cells were then washed 2× in PBS, resuspended at 5×106/ml and injected (200 μl) into the tail vein of recipient mice.

### Radiation:

Splenic leukocytes (5×106/ml) were irradiated (500 rads) using cell irradiator (γ source, Cs-137) and then incubated with IVIg or BSA as described above.

### Induction and Treatment of ITP:

For the administration of IVIg, BSA, or IMPC-cells, mice were first injected intraperitoneally with 50 mg of IVIg, BSA (˜equivalent to 2 g/kg body weight), IMPC cells, or control-IMCP cells. After 24 hrs, mice were rendered thrombocytopenic by the intraperitoneal injection of 2 μg anti-CD41 (anti-integrin αIIb) antibody in 200 μL PBS. Twenty-four hours later, mice were bled by the saphenous vein and the platelets were counted on a flow rate-calibrated FACScan flow cytometer (Becton Dickinson) as previously described in detail (Br. J. Haematol. 115:679-686, 2001; Blood. 101: 708-3713, 2003).

### T Cell Purification:

T cells were purified from spleens by magnetic separation using a T cell negative selection kit (StemCell Technologies, Vancouver, BC) according to manufacturer's instructions. Briefly, splenocytes were prepared in Ca++ and Mg++ free PBS containing 2% heat-inactivated fetal calf serum and 5% normal rat serum at 108 nucleated cells/mL. Splenocytes were then incubated with T cell negative selection cocktail (containing antibodies to CD11b, CD45R, Ly-6G(Gr-1), TER 119) at 20 μl/mL, followed by biotin selection cocktail at 100 μl/mL, and magnetic nanoparticles at 100 μl/mL. All incubations were done for 15 min at 4° C. The recovered cells were stained with anti-CD3-FITC (10 μg/mL) and anti-CD19-PE (4 μg/mL) for 30 min at 4° C., washed, and analyzed by a FACScan flow cytometer. The recovered cells were routinely >90% CD3+ and <1% CD19+.

### Cell Purification:

B cells were purified from the spleen by magnetic separation using a B cell negative selection kit (StemCell Technologies, Vancouver, BC) according to manufacturer's instructions. Briefly, splenocytes were prepared in Ca++ and Mg++ free PBS containing 2% heat-inactivated fetal calf serum and 5% normal rat serum at 108 nucleated cells/mL. Splenocytes were then incubated with mouse FcR blocker (anti-CD16/32) at 10 μl/mL, B cell negative selection cocktail (containing antibodies to CD4, CD8, CD11b, Ly-6G(Gr-1), TER 119) at 20 μl/mL, followed by biotin selection cocktail at 100 μl/mL, and magnetic nanoparticles at 100 μl/mL. All incubations were done for 15 min at 4° C. The recovered cells were stained with anti-CD3-FITC (10 μg/mL) and anti-CD19-PE (4 μg/mL) for 30 min at 4° C., washed, and analyzed by FACScan flow cytometer. The recovered cells were routinely >80% CD19+ and 10% CD3+.

### Results

We found that leukocytes can be treated with IVIg in vitro, washed free of unbound IVIg, and when as little as 106 of these cells are injected into a mouse, an IVIg-like effect is observed (ie. rapid reversal of the autoimmune disease symptom, in ITP, thrombocytopenia). This effect is specifically observed with blood or splenic leukocytes, but not red blood cells. The leukocytes must also be biologically active (ie γ irradiated or paraformaldehyde fixed leukocytes do not work) indicating that simple passive transfer of the IVIg is not the mode of action. B and T cells are not required for this clinical effect of IVIg. Thus, we have strong experimental evidence that the antibody-based treatment regimes of the present invention, induce a priming event in innate leukocytes which endows leukocytes with the ability to ameliorate or inhibit autoimmune disease, specifically in ITP, thrombocytopenia, or in inflammatory arthritis, joint inflammation. We call this effect “IVIg-mediated cellular programming” (IMCP). This term is intended to more broadly refer to an antibody-mediated cellular programming effect, however for simplicity reference is made to the IVIg example, and hence IMCP is used throughout without prejudice. It is not intended to restrict the effect to only IVIg treatment regimes.

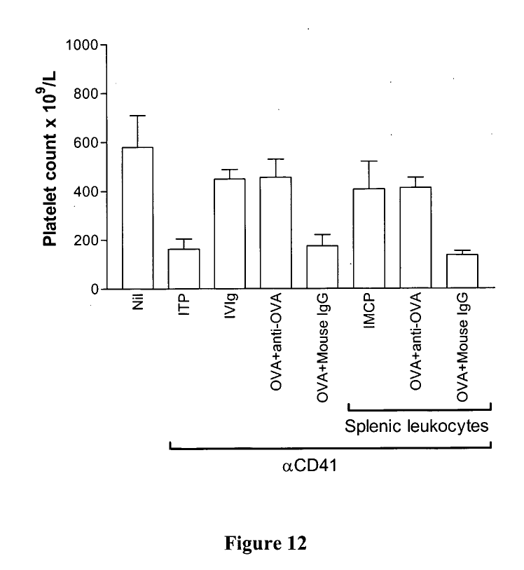

A monoclonal antibody (anti-CD44) is also demonstrated to inhibit immune thrombocytopenia by the same mechanism (ie. an IMCP-like effect in FIG. 12. Here, anti-CD44+ leukocytes were incubated for 30 min, unbound anti-CD44 was washed off, leukocytes were then injected into ITP mice, and an amelioration of thrombocytopenia resulted. Mice in the first column (Nil) were uninjected. Mice in the second column (ITP) were treated with anti-platelet antibody (αCD41) only. On Day 1, mice in the third and fourth column (IMCP) were injected intravenously with splenic leukocytes (106/mouse) that went through the IMCP process with IVIg or anti-CD44 for 30 min. On Day 2 mice in columns (second to fourth) were injected with 2-μg anti-platelet antibody. On Day 3, all mice were bled for platelet enumeration as described (Blood 105:1546-1548, 2005).

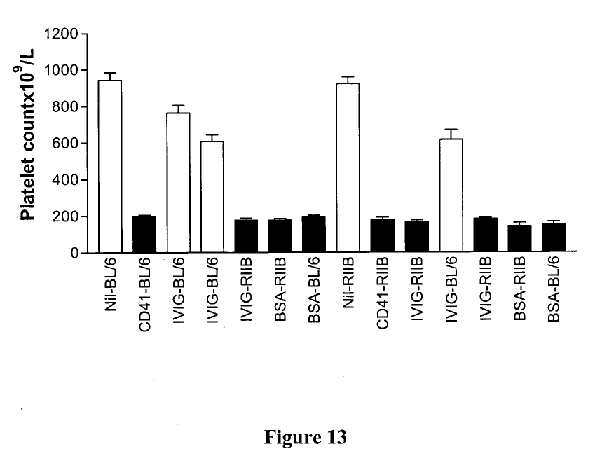

FIG. 13 illustrates an antibody-mediated cellular programming effect, herein referred to as IMCP, as mentioned above, at work in splenic leukocytes incubated with monoclonal anit-OVA, thus establishing a basis for the mode of action of the treatment regimes of the present invention. As illustrated, anti-ovalbumin+ovalbumin+leukocytes are incubated for 30 min, unbound anti-ovalbumin and ovalbumin are washed off, and leukocytes are injected into ITP mice to provide ameliorating effect against thrombocytopenia in vivo. According to FIG. 13, mice in the first column (Nil) were uninjected. Mice in the second column (ITP) were treated with anti-platelet antibody (αCD41) only. On Day 1, mice in the third column (IVIg) were injected with 50 mg/ml of dialyzed IVIg. Mice in the fourth column were injected (i.v.) with 1 mg OVA that had been pre-incubated with 50 μg of monoclonal anti-OVA (IgG1, clone OVA-14 Sigma). Mice in the fifth column were treated as in fourth column except with control mouse IgG (mouse IgG, Cat# 10400, Caltag) in place of monoclonal anti-OVA. Mice in the sixth column (IMCP) were injected intravenously with splenic leukocytes (106/mouse) that went through the IMCP process with dialyzed IVIg for 30 min. Mice in the seventh column were treated with splenic leukocytes (106/mouse) that went through IMCP process with 1 mg OVA that had been pre-incubated with 50 μg of monoclonal anti-OVA for 30 min. Mice in the eigth column were treated as in seventh column except with control mouse IgG in place of monoclonal-anti-OVA. On Day 2, mice in columns (second to eigth) were injected with 2 μg anti-platelet antibody. On Day 3, all mice were bled for platelet enumeration as described (Blood 102:558-560, 2003).

IVIg, anti-CD4 (KM-114), and antibody to soluble antigens (in the presence of the soluble antigen) cannot ameliorate thrombocytopenia in mice which are genetically deficient in the inhibitory Fcγ receptor (FcγRIIB) Interestingly, however, we show here that these same antibodies can, all ameliorate thrombocytopenia when they are pre-incubated with leukocytes isolated from mice that are genetically deficient in FcγRIIB (FcγRIIB^{-/-}) and the FcγRIIB^{-/-} leukocytes are injected into wild type mice. Thus, the IMCP effect as herein reported can work where leukocytes do not express an FcgammaRIIB receptor. Although, FcgammaRIIB receptor expression was required in the recipient in order to achieve IMCP. In the reverse of this experiment (where the leukocytes are from FcγRIIB^{30 /+} mice and the recipient mice are FcγRIIB^{-/-}), again, IVIg, anti-CD44, and anti-soluble antigen (+ the antigen) all cannot ameliorate the thrombocytopenia (FIG. 14). As shown in FIG. 14, mice in the 1^{st} column (Nil-BL/6) are uninjected C57BL/6 mice. Mice in the 2^{nd} column (CD41-BL/6) were C57BL/6 mice treated with anti-platelet antibody (αCD41) only. Mice in the 8^{th} column (Nil-RIIB) were uninjected FcγRIIB^{-/-} mice. Mice in the 9^{th} column (CD41-RIIB) were FcγRIIB^{-/-} mice treated with anti-platelet antibody (αCD41) only. On Day 1, mice in the 3^{rd} column (IVIG-BL/6) were injected with 50 mg/ml IVIg. Mice in the fourth column (IVIG-BL/6) were C57BL/6 mice injected intravenously with splenic leukocytes (10⁶/mouse) from C57BL/6 mice that went through the IMCP process with IVIg for 30 min. Mice in the 5^{th} column (IVIG-RIIB) were FcγRIIB^{-/-} mice injected intravenously with splenic leukocytes (10⁶/mouse) from C57BL/6 mice that went through the IMCP process with IVIg for 30 min. Mice in the 6^{th} column (BSA-RIIB) were FcγRIIB^{-/-} mice injected intravenously with splenic leukocytes (10⁶/mouse) from C57BL/6 mice that went through the IMCP process with BSA for 30 min. Mice in the 7^{th} column (BSA-BL/6) were C57BL/6 mice injected intravenously with splenic leukocytes (10⁶/mouse) from C57BL/6 mice that went through the IMCP process with BSA for 30 min. Mice in the 10^{th} column (IVIG-RIIB) were injected with 50 mg/ml IVIg. Mice in the 11^{th} column (IVIG-BL/6) were C57BL/6 mice injected intravenously with splenic leukocytes (10⁶/mouse) from FcγRIIB^{-/-} mice that went through the IMCP process with IVIg for 30 min. Mice in the 12^{th} column (IVIG-RIIB) were FcγRIIB^{-/-} mice injected intravenously with splenic leukocytes (10⁶/mouse) from FcγRIIB^{-/-} mice that went through the IMCP process with IVIg for 30 min. Mice in the 13^{th} column (BSA-RIIB) were FcγRIIB^{-/-} mice injected intravenously with splenic leukocytes (10⁶/mouse) from FcγRIIB^{-/-} mice that went through the IMCP process with BSA for 30 min. Mice in the 14^{th} column (BSA-RIIB) were C57BL/6 mice injected intravenously with splenic leukocytes (10⁶/mouse) from FcγRIIB^{-/-} mice that went through the IMCP process with BSA for 30 min. On Day 2, mice in columns (2^{nd} to 7^{th} and 9^{th} to 14^{th}, inclusive) were injected with 2 μg anti-platelet antibody. On Day 3, all mice were bled for platelet enumeration as described in Blood 102:558-560, 2003 with the exception that mice were bled by the saphenous vein in accordance with this embodiment of the present invention.

We therefore conclude that IVIg, anti-CD44, and anti-soluble antigen (in the presence of the antigen) do not function by binding to the FcγRIIB on the leukocyte but do all function by a highly related mechanism, which we refer to as an IVIg-mediated cellular programming mechanism, or IMCP. Furthermore, the cellular programming mechanism (IMCP) of the present invention establishes an underlying mode of action for antibody-based treatment regimes of the present invention that appears to be more accurate than the previously reported RES blockade mechanism.

### DISCUSSION

We have observed that antibodies to soluble antigens ameliorated both murine ITP as well as arthritis. Since the immunological mechanisms involved in both of these diseases is very different, i.e. phagocytosis of opsonized platelets in the spleen vs. joint destruction, our data demonstrate that the therapeutic effects of the anti-soluble-antigen regime work to ameliorate autoimmune disease, in general. In addition to the effectiveness of this treatment regime in both ITP and arthritis treatment, we have also established an underlying mechanism of action for the anti-soluble-antigen regime that is common to that of IVIg (the standard therapy for a multitude of automimmune diseases) and anti-CD44 antibody. That is, an antibody-mediated cellular programming effect, as illustrated with pre-incubated leukocytes. Thus, further supporting the potential of an anti-soluble-antigen treatment regime of the present invention in the treatment of a plurality of autoimmune diseases.

The above described antibodies and antibody-antigen and antibody-antigen-cell complexes can be incorporated in pharmaceutical compositions to be injected in the mammal. Such compositions may also comprise a pharmaceutically acceptable carrier as would be known in the art.

The compositions can be injected in the mammal by several routes of administration comprising intravenously, interperitoneally, intradermally, intramuscularly, subcutaneously, orally or rectally.

It will be appreciated by persons skilled in the art that other antigens and antibodies could also be used according to the above described method to achieve similar results. It will also be appreciated that the method and composition could be applied to mammals, other than mice and rabbits, such as humans.

The embodiment(s) of the invention described above is(are) intended to be exemplary only. The scope of the invention is therefore intended to be limited solely by the scope of the appended claims.

1. A method for treating an immune thrombocytopenia or inflammatory arthritis in a mammal by means of an in vivo antibody-antigen interaction, without invoking the biological function of the antigen, which method comprises administering to said mammal an effective amount of at least one IgG antibody and/or a complementary soluble antigen thereof, wherein said administration results in the selective binding of said antibody with said soluble antigen in vivo in said mammal, and wherein said antigen is substantially soluble in vivo.

2. The method according to claim 1 wherein said soluble antigen is a foreign antigen.

3. The method according to claim 2 wherein said soluble foreign antigen is administered to said mammal prior to or following administering said antibody.

4. The method according to claim 2 wherein said soluble foreign antigen and said antibody are incubated together to form antibody-antigen conjugates prior to administering said conjugates to said mammal.

5. The method according to claim 2 wherein said foreign antigen is ovalbumin.

6. The method according to claim 2 wherein said mammal has a pre-existing IgG to said soluble antigen and an effective amount of said soluble antigen is administered.

7. (canceled)

8. The method according to claim 1 wherein said soluble antigen is endogenous.

9. The method according to claim 8 wherein an effective amount of said antibody is administered.

10. The method according to claim 8 wherein said endogenous soluble antigen is obtained from said mammal and incubated with said antibody to form antibody-antigen conjugates, said conjugates being administered to said mammal.

11. The method according to claim 8 wherein said soluble endogenous antigen is selected from albumin, transferring and combinations thereof.

12. (canceled)

13. (canceled)

14. (canceled)

15. (canceled)

16. The method according to claim 1 for treating an immune thrombocytopenia.

17. The method according to claim 1 for treating inflammatory arthritis.

18. A method of inhibiting platelet clearance in a patient in need thereof by means of an in vivo antibody-antigen interaction, without invoking the biological function of the antigen, which method comprises administering to the patient a composition comprising a therapeutic amount of at least one IgG antibody and/or a complementary soluble antigen thereof, and a pharmaceutically acceptable carrier, wherein said administration results in the selective binding of said antibody with said soluble antigen in said patient, and wherein said antigen is substantially soluble in vivo.

19. The method according to claim 18, wherein the therapeutic amount of the at least one antibody ranges from about 0.1 μg to about 1 g per kg of body weight per day.

20. The method according to claim 18, wherein the at least one antibody and/or soluble antigen is administered for a time sufficient to therapeutically increase and maintain platelet cell counts.

21. The method according to claim 18 wherein said soluble antigen is a foreign antigen.

22. The method according to claim 21 wherein said soluble antigen is administered to said mammal prior to or following administering said antibody.

23. The method according to claim 21 wherein said soluble antigen and said antibody are incubated together to form antibody-antigen or antibody-antigen-blood cell conjugates prior to administering said conjugates to said mammal.

24. The method according to claim 21 wherein said soluble antigen is ovalbumin.

25. The method according to claim 21 wherein said mammal has a pre-existing IgG to said soluble antigen and an effective amount of said soluble antigen is administered.

26. (canceled)

27. The method according to claim 18 wherein said soluble antigen is endogenous.

28. The method according to claim 27 wherein said soluble antigen is selected from albumin, transferring and combinations thereof.

29. The method according to claim 27 wherein an effective amount of said antibody is administered.

30. The method according to claim 27 wherein said soluble antigen is obtained from said mammal and incubated with said antibody to form antibody-antigen conjugates, said conjugates being administered to said mammal.

31. (canceled)

32. (canceled)

33. A pharmaceutical composition for treating an immune thrombocytopenia or inflammatory arthritis by means of an in vivo antibody-antigen interaction, without invoking the biological function of the antigen, said composition comprising an effective amount of at least one IgG antibody and/or a complementary soluble antigen thereof in combination with a pharmaceutically acceptable carrier, wherein administration of said composition results in the selective binding of said antibody with said soluble antigen in vivo in said mammal, and wherein said antigen is substantially soluble in vivo.

34. The composition according to claim 33, wherein said antibody and/or soluble antigen is capable of inhibiting platelet clearance.

35. The composition according to claim 33 wherein said soluble antigen is foreign antigen.

36. The composition according to claim 35 wherein said composition comprises said soluble antigen for administration to said mammal prior to or following administering said antibody.

37. The composition according to claim 35 wherein said composition comprises said soluble foreign antigen and said antibody as antibody-antigen or antibody-antigen-blood cell conjugates for administering said conjugates to said mammal.

38. The composition according to claim 35 wherein said foreign antigen is ovalbumin.

39. The composition according to claim 35 wherein said mammal has a pre-existing IgG to said soluble antigen and said composition comprises an effective amount of said soluble antigen.

40. (canceled)

41. The composition according to claim 33 wherein said soluble antigen is endogenous.

42. The composition according to claim 41 wherein said composition comprises an effective amount of said antibody.

43. The composition according to claim 41 wherein said soluble endogenous antigen is selected from albumin, transferring and combinations thereof.

44. The composition according to claim 41 wherein said composition comprises said endogenous soluble antigen obtained from said mammal and said antibody as antibody-antigen conjugates for administering said conjugates to said mammal.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)
